(19) United States
(12) Patent Application Publication
(10) Pub. No.: US 2026/0162825 A1
(43) Pub. Date: Jun. 11, 2026

(54) SYSTEMS AND METHODS FOR GENERATING HEALTH DISORDER SUMMARY AND RECOMMENDATIONS USING LARGE LANGUAGE MODELS

(71) Applicant: Tata Consultancy Services Limited, Mumbai (IN)

(72) Inventors: Arnab CHATTERJEE, New Delhi (IN); Sachin n/a, New Delhi (IN); Vaishali AGGARWAL, New Delhi (IN)

(73) Assignee: Tata Consultancy Services Limited, Mumbai (IN)

(21) Appl. No.: 19/330,948

(22) Filed: Sep. 17, 2025

(30) Foreign Application Priority Data
Dec. 10, 2024 (IN) 202421097230

(51) Int. Cl. G16H 50/20; G16H 10/60; G16H 20/70

(52) U.S. Cl. CPC G16H 50/20; G16H 10/60; G16H 20/70

(57) ABSTRACT

Textual data from social platforms captures various aspects of mental health through discussions around and across issues, while users reach out for help and others sympathize and offer support. Present disclosure leverages Natural Language Processing (NLP) and Generative AI techniques to identify and assess mental health disorders, detect their severity, and create recommendations for behavior change and therapeutic interventions based on input data associated with users. To classify the disorders, the system leverages Large Language Models (LLMs) to filter relevant data from input data, the relevant data pertains to health disorder of users. Further, from the relevant data, various features are extracted to create user profiles and different summaries. The summaries are then aggregated to obtain a final summary for generation of actionable health disorder specific recommendations.

### PRIORITY CLAIM

This U.S. patent application claims priority under 35 U.S.C. § 119 to: India application No. 202421097230, filed on Dec. 10, 2024. The entire contents of the aforementioned application are incorporated herein by reference.

### TECHNICAL FIELD

The disclosure herein generally relates to text analysis, and, more particularly, to systems and methods for generating health disorder summary and recommendations using Large Language Models.

### BACKGROUND

Mental health is crucial for overall well-being, with early detection and intervention being vital. Mental illness affects millions worldwide and is a significant health burden, worsened by the COVID-19 pandemic. Social media offers valuable insights into mental health through extensive user discussions around experiences and struggles. However, analyzing this data is challenging due to informal language, slang, and emojis, which complicate traditional natural language processing (NLP) techniques. Despite these challenges, social media provides important insights and requires efficient, scalable analysis methods.

Generative AI (GenAI), especially Large Language Models (LLMs), has greatly improved the analysis of unstructured text data by understanding and generating human-like text. However, for mental health data, one needs to be careful about misinformation and misdiagnosis due to the potential for generating convincing but false content. Ethical issues, biases, and the need for human oversight are important considerations.

### SUMMARY

Embodiments of the present disclosure present technological improvements as solutions to one or more of the above-mentioned technical problems recognized by the inventors in conventional systems.

For example, in one aspect, there is provided a processor implemented method for generating health disorder summary and recommendations using Large Language Models. The method comprises receiving, via one or more hardware processors, an input data pertaining to a plurality of users; filtering, by using one or more Large Language Models (LLMs) via the one or more hardware processors, the input data to obtain a filtered data specific to at least one health disorder pertaining to at least a subset of the plurality of users; extracting, by using the one or more LLMs via the one or more hardware processors, a first set of features and a second set of features from the filtered data based on one or more context associated prompts; creating, by using the one or more LLMs via the one or more hardware processors, one or more user profiles for the subset of the plurality of users based on the extracted first set of features and the extracted second set of features; generating a first summary and a second summary based on the extracted first set of features and the extracted second set of features respectively for each user profile amongst the one or more user profiles; generating, by using the one or more LLMs via the one or more hardware processors, a third summary based on the first summary and the second summary; generating, by using the one or more LLMs via the one or more hardware processors, one or more actionable health disorder specific recommendations based on an analysis of the third summary.

In an embodiment, the first set of features comprises at least one of (i) a duration, (ii) a frequency, (iii) a recurrence, and (iv) one or more explicit time and date mentions.

In an embodiment, the second set of features comprises at least one of (i) a severity of the at least one health disorder, (ii) one or more factors impacting the at least one health disorder, and (iii) a language and tonality in the filtered data pertaining to the at least one health disorder.

In an embodiment, the one or more actionable health disorder specific recommendations comprise at least one of a therapy, and one or more behavioral changes.

In another aspect, there is provided a processor implemented system for generating health disorder summary and recommendations using Large Language Models. The system comprises: a memory storing instructions; one or more communication interfaces; and one or more hardware processors coupled to the memory via the one or more communication interfaces, wherein the one or more hardware processors are configured by the instructions to receive an input data pertaining to a plurality of users; filter, by using one or more Large Language Models (LLMs), the input data to obtain a filtered data specific to at least one health disorder pertaining to at least a subset of the plurality of users; extract, by using the one or more LLMs, a first set of features and a second set of features from the filtered data based on one or more context associated prompts; create, by using the one or more LLMs, one or more user profiles for the subset of the plurality of users based on the extracted first set of features and the extracted second set of features; generate, by using the one or more LLMs, a first summary and a second summary based on the extracted first set of features and the extracted second set of features respectively for each user profile amongst the one or more user profiles; generate, by using the one or more LLMs, a third summary based on the first summary and the second summary; generate, by using the one or more LLMs, one or more actionable health disorder specific recommendations based on an analysis of the third summary.

In an embodiment, the first set of features comprises at least one of (i) a duration, (ii) a frequency, (iii) a recurrence, and (iv) one or more explicit time and date mentions.

In an embodiment, the second set of features comprises at least one of (i) a severity of the at least one health disorder, (ii) one or more factors impacting the at least one health disorder, and (iii) a language and tonality in the filtered data pertaining to the at least one health disorder.

In an embodiment, the one or more actionable health disorder specific recommendations comprise at least one of a therapy, and one or more behavioral changes.

In yet another aspect, there are provided one or more non-transitory machine-readable information storage mediums comprising one or more instructions which when executed by one or more hardware processors cause generating health disorder summary and recommendations using Large Language Models by receiving an input data pertaining to a plurality of users; filtering, by using one or more Large Language Models (LLMs), the input data to obtain a filtered data specific to at least one health disorder pertaining to at least a subset of the plurality of users; extracting, by using the one or more LLMs, a first set of features and a second set of features from the filtered data based on one or more context associated prompts; creating, by using the one or more LLMs, one or more user profiles for the subset of the plurality of users based on the extracted first set of features and the extracted second set of features; generating, by using the one or more LLMs, a first summary and a second summary based on the extracted first set of features and the extracted second set of features respectively for each user profile amongst the one or more user profiles; generating, by using the one or more LLMs, a third summary based on the first summary and the second summary; generating, by using the one or more LLMs, one or more actionable health disorder specific recommendations based on an analysis of the third summary.

In an embodiment, the first set of features comprises at least one of (i) a duration, (ii) a frequency, (iii) a recurrence, and (iv) one or more explicit time and date mentions.

In an embodiment, the second set of features comprises at least one of (i) a severity of the at least one health disorder, (ii) one or more factors impacting the at least one health disorder, and (iii) a language and tonality in the filtered data pertaining to the at least one health disorder.

In an embodiment, the one or more actionable health disorder specific recommendations comprise at least one of a therapy, and one or more behavioral changes.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 depicts an exemplary system for generating health disorder summary and recommendations using Large Language Models, in accordance with an embodiment of the present disclosure.

FIG. 2 depicts an exemplary high level block diagram of the system of FIG. 1 for generating health disorder summary and recommendations using Large Language Models, in accordance with an embodiment of the present disclosure.

FIG. 3 depicts an exemplary flow chart illustrating a method for generating health disorder summary and recommendations using Large Language Models of the systems of FIG. 1-2, in accordance with an embodiment of the present disclosure.

FIG. 4 depicts a graphical representation illustrating used language styles and tones by online users, in accordance with an embodiment of the present disclosure.

FIG. 5 depicts a graphical representation illustrating distribution of various therapies recommended to the users, in accordance with an embodiment of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

Analyzing social media data for mental health diagnosis has become increasingly significant due to the wealth of information available on social media platforms such as Twitter®, Reddit®, etc. The field has seen significant advancements, with various methodologies developed to enhance the accuracy and efficiency of identifying mental health conditions from user generated content. Linguistic markers of depression examined from Twitter data highlighted the potential of social media for mental health insights. Recent work on identifying stress factors that lead to mental health decline has demonstrated the effectiveness of NLP techniques in this domain. Various machine learning techniques have been used to detect mental health issues on social media platforms, where deep learning methods enhanced detection accuracy. “Dreaddit”, a dataset for stress analysis in social media posts, was used to demonstrate the efficacy of NLP techniques for identifying stress-related content on Reddit using binary classification.

Another literature work discussed a gold standard dataset for detecting depression levels from social media posts using traditional algorithms and data augmentation in a multi-class classification framework. Bidirectional Encoder Representations from Transformers (BERT) has set new NLP benchmarks by employing bidirectional transformer training to better understand language context than earlier models. Yet another literature, ROBERTa (Robustly optimized BERT approach) improves upon BERT by optimizing the pre-training phase. Analyzing the literature, it is found that LLMs resemble low-quality therapy behaviors but also reflect more on clients' needs and strengths, indicating the need for further research to ensure high-quality care.

Embodiments of the present disclosure provide a system and a method that leverage the knowledge of Large Language Models (LLMs) to (i) identify mental health issues by utilizing social media data to detect whether it is related to mental health, (ii) classify disorders and severity, and (iii) recommend interventions for (i) behavior changes and (ii) therapies based on the identified disorders and their severity. More specifically, the system and method of the present disclosure address the lack of accessible, scalable, and cost-effective mental health support by leveraging LLMs to provide early detection, diagnosis using widely accepted international psychiatric standards, and personalized recommendations. Using mental health related user text as input, the system automatically generates different features along with recommendations to be used by mental health practitioners as well as organizations. It benefits practitioners by aiding in diagnosis and reducing workload, while helping corporations by enhancing employee wellbeing, providing personalized support, and offering data-driven insights, leading to a healthier and more proactive work environment, thereby positively impacting productivity.

This addresses gaps in automated, personalized mental health assessment by integrating both temporal and non-temporal information/features for assessment using international Psychiatric standards (e.g., DSM5), from text inputs. The system of the present disclosure provides context-aware, scalable recommendations and ensures privacy, improving the accuracy and relevance of mental health support while overcoming limitations in current/conventional solutions.

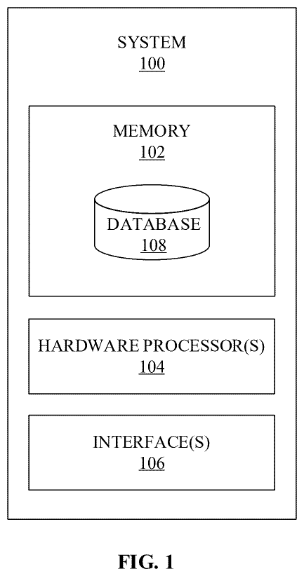

Referring now to the drawings, and more particularly to FIGS. 1 through 5, where similar reference characters denote corresponding features consistently throughout the figures, there are shown preferred embodiments, and these embodiments are described in the context of the following exemplary system and/or method.

FIG. 1 depicts an exemplary system 100 for generating health disorder summary and recommendations using Large Language Models, in accordance with an embodiment of the present disclosure. In an embodiment, the system 100 includes one or more hardware processors 104, communication interface device(s) or input/output (I/O) interface(s) 106 (also referred as interface(s)), and one or more data storage devices or memory 102 operatively coupled to the one or more hardware processors 104. The one or more processors 104 may be one or more software processing components and/or hardware processors. In an embodiment, the hardware processors can be implemented as one or more microprocessors, microcomputers, microcontrollers, digital signal processors, central processing units, state machines, logic circuitries, and/or any devices that manipulate signals based on operational instructions. Among other capabilities, the processor(s) is/are configured to fetch and execute computer-readable instructions stored in the memory. In an embodiment, the system 100 can be implemented in a variety of computing systems, such as laptop computers, notebooks, hand-held devices (e.g., smartphones, tablet phones, mobile communication devices, and the like), workstations, mainframe computers, servers, a network cloud, and the like.

The I/O interface device(s) 106 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite. In an embodiment, the I/O interface device(s) can include one or more ports for connecting a number of devices to one another or to another server.

The memory 102 may include any computer-readable medium known in the art including, for example, volatile memory, such as static random-access memory (SRAM) and dynamic-random access memory (DRAM), and/or non-volatile memory, such as read only memory (ROM), erasable programmable ROM, flash memories, hard disks, optical disks, and magnetic tapes. In an embodiment, a database 108 is comprised in the memory 102, wherein the database 108 comprises input data received from one or more sources (e.g., social media, and the like). The input data pertains to health information of a plurality of users. The database 108 further comprises filtered data that is specific to health disorders of one or more users, and the like. The memory 102 further comprises (or may further comprise) information pertaining to input(s)/output(s) of each step performed by the systems and methods of the present disclosure. In other words, input(s) fed at each step and output(s) generated at each step are comprised in the memory 102 and can be utilized in further processing and analysis.

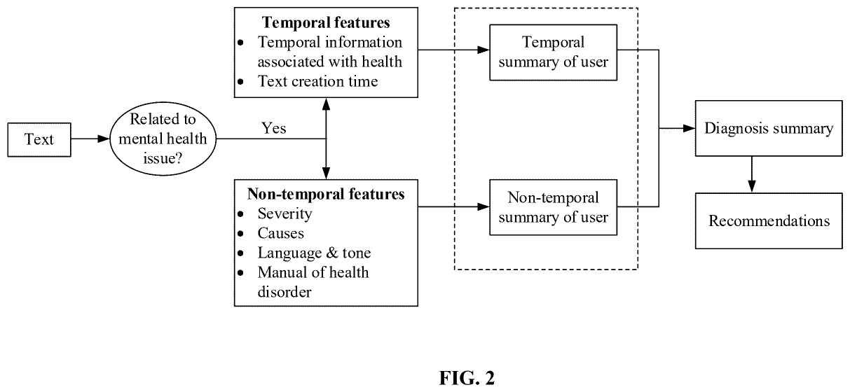

FIG. 2, with reference to FIG. 1, depicts an exemplary high level block diagram of the system 100 of FIG. 1 for generating health disorder summary and recommendations using Large Language Models, in accordance with an embodiment of the present disclosure.

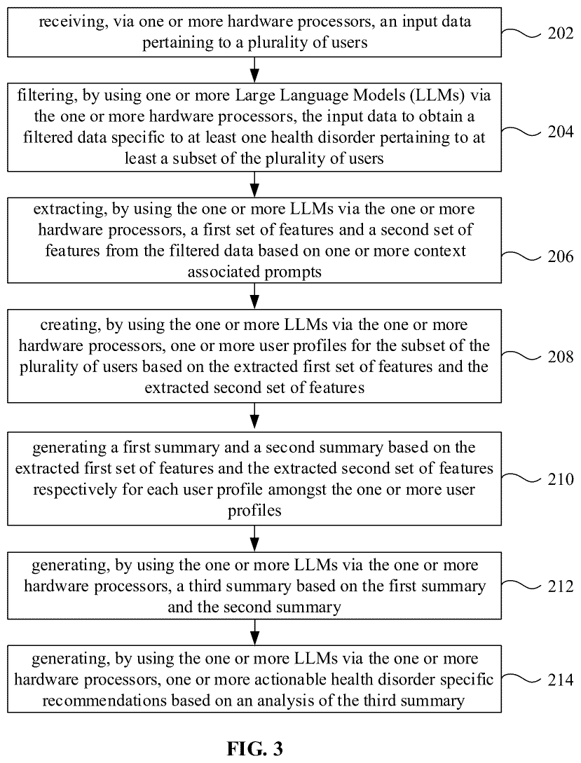

FIG. 3, with reference to FIGS. 1-2, depicts an exemplary flow chart illustrating a method for generating health disorder summary and recommendations using Large Language Models of the systems 100 of FIG. 1-2, in accordance with an embodiment of the present disclosure. In an embodiment, the system(s) 100 comprises one or more data storage devices or the memory 102 operatively coupled to the one or more hardware processors 104 and is configured to store instructions for execution of steps of the method by the one or more processors 104. The steps of the method of the present disclosure will now be explained with reference to components of the system 100 of FIG. 1, the block diagram of the system 100 depicted in FIG. 2, and the flow diagram as depicted in FIG. 3. Although process steps, method steps, techniques or the like may be described in a sequential order, such processes, methods, and techniques may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

At step 202 of the method of the present disclosure, the one or more hardware processors 104 receive an input data pertaining to a plurality of users. The input data may comprise at least one of one or more posts, one or more comments from the plurality of users. This input data may be obtained through various one or more sources such as websites, community forums, social media, and the like. Below Table 1 depicts exemplary input data received from the one or more sources:

TABLE 1

| Text | Author/User | Time/date |
|---|---|---|
| It can which is why I | jay-sid- | 2020 Apr. 3 05:45:20 |
| have two accounts and | | |
| only on this one I follow | | |
| the depression thread. | | |
| The ones that make | | |
| most depressed is | | |
| when people post about | | |
| how their life is getting | | |
| amazing and it gets A | | |
| bunch of upvotes. Like | | |
| that's great you're | | |
| getting better but I | | |
| haven't been able to in | | |
| 5 years and just don't | | |
| want to see that | | |
| well, if you do tell a | testamentsofanguish | 2019 Dec. 2 11:57:58 |
| teacher or friend, tell | | |
| them how you feel | | |
| about others as well. | | |
| why you don't like | | |
| reaching out. maybe | | |
| that'd help them | | |
| understand your | | |
| feelings more. but it's | | |
| entirely up to you. i | | |
| wish you the best | | |
| regardless. | | |
| great job! | testamentsofanguish | 2019 Dec. 15 23:46:34 |
| i am so lonely and you | d3ad1ns1de | 2020 May 2 01:37:52 |
| would think with the | | |
| internet there wouldn't | | |
| be anymore loneliness. | | |
| but yet here i am 39, no | | |
| friends, no family, | | |
| playing world of | | |
| warcraft and absolutely | | |
| no one to spend time | | |
| with or talk too and | | |
| absolutely alone even | | |
| in a mmo. didn't think it | | |
| was possible but surely | | |
| it is. the loneliness is | | |
| killing me, seeing all | | |
| these couples having | | |
| someone with them | | |
| and doing things with | | |
| and sharing things with | | |
| and yet here i sit can't | | |
| even get a simple date | | |
| or even a woman who | | |
| is interested in talking | | |
| to me that doesn't | | |
| ghost me after an hour | | |
| or two or a day or two. i | | |
| am so tired of the | | |
| loneliness. its | | |
| destroying me and | | |
| playing with my mind | | |
| badly. | | |
| was feeling this the | SmarmySlayer | 2020 Feb. 20 06:18:46 |
| past few days, made | | |
| some posts, deleted | | |
| them, did some other | | |
| things and realise what | | |
| i'm feeling isn't normal | | |
| and i'm not faking it to | | |
| myself | | |
| is there a way to die | SubstanceFederal | 2020 Jul. 29 15:11:50 |
| without causing family | | |
| and friends plain? | | |
| you don't need to feel | kindofbrill | 2020 Jun. 23 16:49:52 |
| lonely. we are there to | | |
| make you feel the | | |
| complete opposite. we | | |
| are always here to | | |
| support and help you, | | |
| no matter what. | | |
| whenever you feel | | |
| comfortable, sending | | |
| myself a dm and we'll | | |
| talk. :) | | |
| that's amazing! ahhh | ambition786 | 2020 Mar. 7 16:50:10 |
| i'm so happy for you! :) | | |
| sleep issues? idk i | ambition786 | 2020 Jul. 19 09:47:25 |
| woke up feeling upset | | |
| and sick. just mad at | | |
| myself. honestly this | | |
| happens every single | | |
| time i nap and has | | |
| been a thing since i | | |
| was a kid. short periods | | |
| of sleep tick me off. is | | |
| this odd? feels odd. | | |
| it's hard to work on | carter_pride | 2020 Jun. 1 2:44:44 |
| things i like or want to | | |
| do i don't think i've ever | | |
| stuck to any long term | | |
| project that i wanted to | | |
| do or liked for myself. | | |
| most of the time, i've | | |
| only ever done things | | |
| that i had to do in life. i | | |
| am not sure i could | | |
| ever do anything for | | |
| myself and then stay | | |
| committed to it. that's | | |
| the life of a slave, a | | |
| soldier, and an | | |
| oppressed immigrant. | | |
| and i am not sure i can | | |
| snap out of it. maybe | | |
| it's time to see a | | |
| therapist after all. | | |

At step 204 of the method of the present disclosure, the one or more hardware processors 104 filter, by using the one or more LLMs, the input data to obtain a filtered data specific to at least one health disorder pertaining to at least a subset of the plurality of users. The system 100 employs LLMs (Large Language Model) to filter out irrelevant content, thereby refining the input data to include only posts that are genuinely related to health conditions/mental health/health disorder. Each post is evaluated to determine whether it pertained to health issue, with the LLMs instructed to provide responses one or more pre-defined formats (e.g., say “yes” or “no” format). By leveraging the advanced capabilities of LLMs, the system 100 efficiently classifies/filters the vast number of posts, ensuring the integrity and focus of the analysis on meaningful health-related content. Below Table 2 is the filtering done to identify mental health related post using one or more designed prompt to obtain filtered data way of examples.

TABLE 2

| | | | Related |
|---|---|---|---|
| | System Prompt | User Prompt | to health |
| Text | for Filtration | for Filtration | disorder |
| It can which is | You are an | Problem: Text. | Yes |
| why I have two | advanced AI | Based on this | |
| accounts and | language model | information, | |
| only on this one I | trained to | do you think this | |
| follow the | classify and | problem is | |
| depression | understand | related to a | |
| thread. The | various human | mental disorder? | |
| ones that make | problems. Your | Only give ‘YES’ | |
| most depressed | task is to | or ‘NO’ | |
| is when people | determine | answers nothing | |
| post about how | whether a | else. | |
| their life is | described | | |
| getting amazing | problem is | | |
| and it gets A | related to a | | |
| bunch of upvotes. | mental disorder | | |
| Like that's great | or not. Use your | | |
| you're getting | knowledge of | | |
| better but I | psychology, | | |
| haven't been | mental health, | | |
| able to in 5 years | and diagnostic | | |
| and just don't | criteria such as | | |
| want to see that | those found | | |
| i feel so | in the DSM-5 | | Yes |
| disconnected | (Diagnostic and | | |
| with reality I | Statistical | | |
| don't know how | Manual of Mental | | |
| to explain it but I | Disorders) to | | |
| almost feel like | make your | | |
| everything is a | determination. | | |
| dream at this | Be sensitive and | | |
| point or just not | respectful in your | | |
| real? Memories | analysis, | | |
| from the past | recognizing the | | |
| feel like they | complexity and | | |
| happened in a | nuances of | | |
| different lifetime, | mental health | | |
| really strange | issues. | | |
| feeling | | | |
| exactly! thanks | | | No |
| man, i hope the | | | |
| same for you | | | |
| tonight's going to | | | No |
| be rough | | | |
| I know . . . and it's | | | Yes |
| so scary . . . you | | | |
| Really feel like | | | |
| you're the only | | | |
| human being in | | | |
| this world | | | |
| the realization | | | Yes |
| that life can be | | | |
| taken from you | | | |
| at any moment is | | | |
| the reason i'm | | | |
| unable to enjoy | | | |
| anything | | | |
| tonight's going to | | | No |
| be rough | | | |
| it helps me but | | | No |
| also can make | | | |
| things worse for | | | |
| me. | | | |

At step 206 of the method of the present disclosure, the one or more hardware processors 104 extract, by using the one or more LLMs, a first set of features and a second set of features from the filtered data based on one or more context associated prompts. The first set of features and the second set of features are different from each other, in one embodiment of the present disclosure. For instance, the first set of features comprises temporal features such (i) a duration, (ii) a frequency, (iii) a recurrence, and (iv) one or more explicit time and date mentions, or combinations thereof. The second set of features comprises (i) a severity of the at least one health disorder, (ii) one or more factors impacting the at least one health disorder, (iii) a language and tonality in the filtered data pertaining to the at least one health disorder, (iv) illness as mentioned in a health disorder manual (e.g., Diagnostic and Statistical Manual of Mental Disorders, and the like). The temporal and non-temporal features are extracted using one or more designed prompts which are provided below in Tables 3 through Table 6 along with the examples.
1. Temporal Features: Timeline
2. Non-Temporal Features: DSM-5 Disorder Classification, Severity, Language Style and Tone, Causes, and the like.

TABLE 3

| | System Prompt | User Prompt | | |
|---|---|---|---|---|
| | for DSM-5 | for DSM-5 | | |
| | Disorder | Disorder | | |
| | Classi- | Classi- | DSM-5 | |
| | fication | fication | Disorder | |
| | and Time- | and Time- | Classi- | Time- |
| Text | line | line | fication | line |
| cried in the | You are an | Analyze the | Major | No |
| car today | expert in | following text | Depressive | Time- |
| infront of my | clinical | to predict the | Disorder, | line |
| mom I | psychology, | potential | Borderline | |
| canÔÇÖt | specialized in | mental | Personality | |
| even control | diagnosing | health | Disorder | |
| my emotions | mental health | conditions | | |
| anymore. In | conditions | the writer | | |
| the past I | based on | might be | | |
| could but now | textual | suffering | | |
| itÔÇÖs | analysis. | from, based | | |
| like I | Your task is | on DSM-5 | | |
| donÔÇÖt | to predict the | criteria. Also, | | |
| even care I | potential | identify any | | |
| just cry at | mental health | references to | | |
| work, | problems(one | time periods | | |
| around | or more) the | that indicate | | |
| family. The | writer of the | the | | |
| advice | provided text | frequency or | | |
| people give | is most likely | duration of | | |
| me is just Itll | and | the | | |
| get better | prominently | symptoms. | | |
| and that | suffering | Do not | | |
| everyone is | from, | provide any | | |
| sad. As if | following the | explanations | | |
| that makes | DSM- | or reasons. | | |
| me feel | 5(Diagnostic | Provide the | | |
| better. It | and | output in the | | |
| sucks when | Statistical | specified | | |
| the people | Manual of | output format | | |
| closest to | Mental | only. Writer's | | |
| you truly | Disorders, | text: Text | | |
| donÔÇÖt | Fifth Edition) | | | |
| understand | criteria. | | | |
| what | Provide only | | | |
| itÔÇÖs | the names of | | | |
| like living | the problems | | | |
| with this | that are | | Generalized | Last |
| Meds? Does | MOST | | Anxiety | few |
| anyone know | RELEVANT | | Disorder, | weeks |
| a good over | AND | | Adjustment | |
| the counter | STRONGLY | | Disorder | |
| med to help | SUPPORTED | | | |
| calm down? I | by the | | | |
| having the | content in the | | | |
| roughest | text, keeping | | | |
| time in years | your | | | |
| just trying to | conclusions | | | |
| keep myself | within the | | | |
| grounded | framework of | | | |
| and it's been | DSM-5 | | | |
| getting | criteria. In | | | |
| worse over | case, some | | | |
| the last few | of the | | | |
| weeks. | problems can | | | |
| | be combined | | | |
| | into one | | | |
| | major class, | | | |
| | return the | | | |
| | name of the | | | |
| | major class in | | | |
| | the output. | | | |
| | Do not give | | | |
| | any | | | |
| | explanations | | | |
| | or reasons. | | | |
| | Additionally, | | | |
| | you must | | | |
| | identify any | | | |
| | mention of | | | |
| | time periods | | | |
| | that indicate | | | |
| | how often the | | | |
| | condition | | | |
| | occurs, how | | | |
| | long it has | | | |
| | been | | | |
| | prevailing, or | | | |
| | any other | | | |
| | relevant | | | |
| | temporal | | | |
| | information. If | | | |
| | no clear time | | | |
| | frame is | | | |
| | present, give | | | |
| | ‘No Timeline’ | | | |
| | as an output. | | | |
| | Do not give | | | |
| | any | | | |
| | explanations | | | |
| | or reasons. | | | |
| | Output | | | |
| | Format: - If | | | |
| | No timeline is | | | |
| | mentioned: | | | |
| | Potential | | | |
| | Mental health | | | |
| | conditions - | | | |
| | names of the | | | |
| | mental health | | | |
| | problems | | | |
| | predicted | | | |
| | separated by | | | |
| | commas; | | | |
| | Timeline - No | | | |
| | Timeline - If | | | |
| | timeline is | | | |
| | mentioned: | | | |
| | Potential | | | |
| | Mental health | | | |
| | conditions - | | | |
| | names of the | | | |
| | mental health | | | |
| | problems | | | |
| | predicted | | | |
| | separated by | | | |
| | commas; | | | |
| | Timeline - | | | |
| | timeline | | | |
| | period | | | |
| | mentioned. | | | |

TABLE 4

| | System Prompt | User Prompt | |
|---|---|---|---|
| | for Severity | for Severity | |
| Text | Classification | Classification | Severity |
| cried in the car | You are a highly | Here is the | Moderate |
| today infront of | experienced | description of a | |
| my mom I | psychiatrist. Your | user's mental | |
| canÔÇÖt even | task is to | health problem: | |
| control my | examine the | Text. Choose | |
| emotions | given user | one from ‘mild’, | |
| anymore. In the | mental health | ‘moderate’, or | |
| past I could but | problem's | ‘severe’. | |
| now itÔÇÖs | severity | | |
| like I | considering | | |
| donÔÇÖt even | DSM- | | |
| care I just cry at | 5(Diagnostic and | | |
| work, around | | | |
| family. The | Statistical | | |
| advice people | Manual of Mental | | |
| give me is just Itll | Disorders, Fifth | | |
| get better and | Edition) and | | |
| that everyone is | classify it into | | |
| sad. As if that | one of three | | |
| makes me feel | labels: ‘mild’, | | |
| better. It sucks | ‘moderate’, or | | |
| when the people | ‘severe’. | | |
| closest to you | “Respond strictly | | |
| truly donÔÇÖt | with one of these | | |
| understand what | labels without | | |
| itÔÇÖs like | providing any | | |
| living with this | additional | | |
| Meds? Does | details. Only | | Moderate |
| anyone know a | respond with the | | |
| good over the | labels ‘mild’, | | |
| counter med to | ‘moderate’, or | | |
| help calm down? | ‘severe’. | | |
| I having the | | | |
| roughest time in | | | |
| years just trying | | | |
| to keep myself | | | |
| grounded and it's | | | |
| been getting | | | |
| worse over the | | | |
| last few weeks. | | | |

TABLE 5

| | System Prompt | User Prompt | |
|---|---|---|---|
| | for Language | for Language | |
| Text | Style and Tone | Style and Tone | Severity |
| cried in the car | You are an | Text: | Despondent |
| today infront of | expert | Text.\textback- | and |
| my mom I | language | slash{ }n | Frustrated |
| canÔÇÖt | model | Analyze the following | |
| even control | designed to | text and identify the | |
| my emotions | analyze the | overall language style | |
| anymore. In | language style | and tone present | |
| the past I could | and tone of the | throughout and then | |
| but now | text provided. | summarize it in one | |
| itÔÇÖs | Your task is to | line with accurate or | |
| like I | analyze the | the most related | |
| donÔÇÖt | given text to | words.\n Do not | |
| even care | determine the | Provide any other | |
| I just cry | major language | explanations or | |
| at work, | style and tone | reasons. Just give on | |
| around family. | used throughout. | point answer. | |
| The advice | Focus on | | |
| people give me | identifying | | |
| is just Itll get | patterns of | | |
| better and that | language that | | |
| everyone is | indicate the | | |
| sad. As if that | writer's | | |
| makes me feel | emotional and | | |
| better. It sucks | mental state. | | |
| when the | Provide the | | |
| people closest | accurate or the | | |
| to you truly | most related | | |
| donÔÇÖt | words for those | | |
| understand | in the format - | | |
| what | The overall | | |
| itÔÇÖs | language style | | |
| like living with | and tone used | | |
| this | is: [answer] | | Informal |
| Meds? Does | | | and |
| anyone know a | | | Anxious |
| good over the | | | |
| counter med to | | | |
| help calm | | | |
| down? I having | | | |
| the roughest | | | |
| time in years | | | |
| just trying to | | | |
| keep myself | | | |
| grounded and | | | |
| it's been | | | |
| getting worse | | | |
| over the last | | | |
| few weeks. | | | |

TABLE 6

| | System Prompt | User Prompt | |
|---|---|---|---|
| Text | for Causes | for Causes | Severity |
| cried in the car | You are an | Text: Text’. \n | emotional |
| today infront of | advanced | Please analyze | dysregulation, |
| my mom I | language model | the text and | lack of |
| canÔÇÖt even | with expertise in | identify the | emotional |
| control my | mental health | major/potential | control, |
| emotions | diagnosis and | factors that | stress, social |
| anymore. In the | DSM-5 criteria. | might trigger | isolation, |
| past I could but | Your task is to | the mental | lack of |
| now itÔÇÖs | extract and | health disorder | understanding |
| like I | summarize the | described. Base | and support |
| donÔÇÖt | major factors | your analysis | from others, |
| even care | that triggers the | on DSM-5 | and feelings |
| I just cry at | mental health | criteria and | of being |
| work, around | disorder | provide a | misunderstood. |
| family. The | described in the | concise | |
| advice people | text. Consider | summary of | |
| give me is just Itll | symptoms, | these factors | |
| get better and | context, and any | apart from the | |
| that everyone is | references to | disorders.\n Do | |
| sad. As if that | factors triggering | not provide any | |
| makes me feel | mentioned in the | reasons or | |
| better. It sucks | text.\n Do not | explanations. | |
| when the people | provide any | Directly | |
| closest to you | reasons or | provide the | |
| truly donÔÇÖt | explanations. | factors | |
| understand what | Directly provide | triggering | |
| itÔÇÖs | the factors apart | separated | stress, |
| like living | from the disorder | by commas | anxiety, |
| with this | triggering the | without | lack of |
| Meds? Does | mental health | any other | grounding, |
| anyone know a | disorder | reason or | worsening |
| good over the | separated by | explanations. | over time, |
| counter med to | commas without | | recent weeks. |
| help calm down? | any other reason | | |
| I having the | or explanations | | |
| roughest time in | in the format - | | |
| years just trying | The possible | | |
| to keep myself | factors are: | | |
| grounded and it's | [answer] | | |
| been getting | | | |
| worse over the | | | |
| last few weeks. | | | |

At step 208 of the method of the present disclosure, the one or more hardware processors 104 create, by using the one or more LLMs, one or more user profiles for the subset of the plurality of users based on the extracted first set of features and the extracted second set of features. The user profile creation for each user was done using python codes, in one example embodiment of the present disclosure. Below Table 7 depicts the user profile creation by the system 100 by way of examples:

TABLE 7

| | | | | | | Language | |
|---|---|---|---|---|---|---|---|
| | | | DSM-5 | | | Style | |
| Text | Author | datetime | Disorder | Severity | Timeline | and Tone | Causes |
| Text 1 | Author 1 | Datetime 1 | Disorders | Severity | Timeline | Langauge | Causes |
| | | | classified | level | 1/No | Style | identified |
| | | | using the | classified | Timeline | and tone | |
| | | | above | using the | | identified | |
| | | | prompts | above | | | |
| | | | | prompts | | | |
| Text 2 | Author 1 | Datetime 2 | Disorders | Severity | Timeline | Langauge | Causes |
| | | | classified | level | 2/No | Style | identified |
| | | | using the | classified | Timeline | and tone | |
| | | | above | using the | | identified | |
| | | | prompts | above | | | |
| | | | | prompts | | | |
| Text 3 | Author 1 | Datetime 3 | Disorders | Severity | Timeline | Langauge | Causes |
| | | | classified | level | 3/No | Style | identified |
| | | | using the | classified | Timeline | and tone | |
| | | | above | using the | | identified | |
| | | | prompts | above | | | |
| | | | | prompts | | | |
| . | . | . | . | . | . | . | . |
| . | . | . | . | . | . | . | . |
| . | . | . | . | . | . | . | . |
| Text n | Author 1 | Datetime n | Disorders | Severity | Timeline | Langauge | Causes |
| | | | classified | level | n/No | Style | identified |
| | | | using the | classified | Timeline | and tone | |
| | | | above | using the | | identified | |
| | | | prompts | above | | | |
| | | | | prompts | | | |

At step 210 of the method of the present disclosure, the one or more hardware processors 104 generate, by using the one or more LLMs, a first summary (e.g., temporal summary of the user and may be interchangeably used herein) and a second summary (e.g., non-temporal summary of the user and may be interchangeably used herein based on the extracted first set of features and the extracted second set of features respectively for each user profile amongst the one or more user profiles. Below Table 8 through Table 11 depict examples of the first summary and the second summary generated by the system 100.

TABLE 8
Non-Temporal Summary: The input text to generate a
non-temporal summary for Author1 is shown below:

| | | | | | Language | |
|---|---|---|---|---|---|---|
| | | | DSM-5 | | Style | |
| Text | Author | Tag | Disorder | Severity | and Tone | Causes |
| Text | Author | post/ | Disorders | Severity | Langauge | Causes |
| 1 | 1 | comment | classified | level | Style and | iden- |
| | | | using the | classified | tone | tified |
| | | | above | using the | identified | |
| | | | prompts | above | | |
| | | | | prompts | | |
| Text | Author | post/ | Disorders | Severity | Langauge | Causes |
| 2 | 1 | comment | classified | level | Style and | iden- |
| | | | using the | classified | tone | tified |
| | | | above | using the | identified | |
| | | | prompts | above | | |
| | | | | prompts | | |
| Text | Author | post/ | Disorders | Severity | Langauge | Causes |
| 3 | 1 | comment | classified | level | Style and | iden- |
| | | | using the | classified | tone | tified |
| | | | above | using the | identified | |
| | | | prompts | above | | |
| | | | | prompts | | |
| . | . | . | . | . | . | . |
| . | . | . | . | . | . | . |
| . | . | . | . | . | . | . |
| Text | Author | post/ | Disorders | Severity | Langauge | Causes |
| n | 1 | comment | classified | level | Style and | iden- |
| | | | using the | classified | tone | tified |
| | | | above | using the | identified | |
| | | | prompts | above | | |
| | | | | prompts | | |

TABLE 9

| System Prompt to | User Prompt to | Output (Non- |
|---|---|---|
| generate Non- | generate Non- | Temporal Summary |
| Temporal Summary | Temporal Summary | generated) |
| Analyze the dataset | Dataset : Dataframe.\n | {“Overall Severity”: |
| provided, which | Given the | “Severe”, |
| includes the following | dataset with the | “Major Causes”: |
| features: | following columns: | “Childhood trauma, |
| full_text (content), Tag | full_text, Tag, Datetime, | family dynamics, |
| (post or comment), | Disorders, | repeated rejection, |
| Datetime (reporting | Severity, Language | poverty, homelessness, |
| date and | Style and Tone, and | lack of education, and |
| time), Symptoms | Causes, analyze the | social isolation”, |
| (potential mental health | information to produce | “Overall Language |
| disorders), Severity | a summary of the | Style and Tone”: |
| (severity | user's mental | “Despondent, |
| level based on content | health condition.\n 1. | despairing, and |
| and disorders), and | Identify key mental | hopeless, with a focus |
| Timeline (temporal | health disorders | on self-referential |
| information). | mentioned in the | content”, |
| Based on these | Disorders column and | “Recurring Themes”: |
| features and the DSM- | compare them | “Themes of rejection, |
| 5 criteria, generate a | against DSM-5 | isolation, and |
| comprehensive | criteria.\n 2. Assess the | hopelessness are |
| summary of the user's | severity levels provided | prevalent throughout |
| mental health condition. | and related them | the content”, |
| In The Output should | to the potential impact | “DSM-5 Criteria”: “The |
| include the following | on the user's | author's symptoms |
| headers: 1. Severity\n | overall mental health.\n | align with DSM-5 |
| 2. Causes\n 3. | 3. Evaluate the | criteria for Major |
| Language Style | temporal data to | Depressive Disorder, |
| and Tone 4. Recurring | highlight trends, | PTSD, and Adjustment |
| Themes\n 5. Mental | including any worsening | Disorder”, |
| Health Disorders(DSM- | or improvement | “Mental Health |
| 5 Criteria)\n | in the user's condition | Status”: “The author's |
| 6. Mental | over time, analyzing | mental health condition |
| Health Status\n\n | trends over time.\n | appears to be chronic |
| Ensure that the | 4. Detect any pattern | and worsening, with a |
| summary is concise, | or significant | high risk of relapse”} |
| comprehensive, | behavioral changes | |
| presented only in json | indicated by the | |
| format and limited to a | Severity and Datetime | |
| maximum of | columns.\n 5. Identify | |
| 200 words. The | the causes for | |
| summary should not | mental health disorder | |
| consist any additional | inferred from the | |
| Note and any | user's content.\n 6. | |
| other information in the | Analysis of language | |
| beginning or at the end. | style, tone, and | |
| | significant patterns.\n | |
| | The summary should | |
| | be structured, | |
| | cohesive, and suitable | |
| | for further clinical | |
| | evaluation or | |
| | therapeutic | |
| | recommendations. | |

### Input Text for Temporal Summary for Author1:

All the entries do not contain Timeline information and some of them were marked as ‘No Timeline’ in ‘Timeline’ column of Author1 dataframe. For temporal summary, the rows for which the values were marked as ‘No Timeline’ were removed from the dataset and the remaining dataset was given as the input text. The input dataframe for Temporal Summary is as follows:

TABLE 10

| Text | Author | datetime | Tag | Timeline |
|---|---|---|---|---|
| Text 1 | Author1 | datetime1 | post/comment | Timeline 1 |
| Text 2 | Author1 | datetime2 | post/comment | Timeline 2 |
| Text 3 | Author1 | datetime3 | post/comment | Timeline 3 |
| . | . | . | . | . |
| . | . | . | . | . |
| . | . | . | . | . |
| Text k | Author1 | datetimek | post/comment | Timeline k |

TABLE 11

| System Prompt to | User Prompt to | |
|---|---|---|
| generate Temporal | generate Temporal | Output (Temporal |
| Summary | Summary | Summary generated) |
| You are an expert in | Dataset: Dataframe.\n | “The user, |
| mental health analysis | Analyze the | beauteeedur, has |
| with a focus on | dataset provided, which | posted a total of 5 |
| temporal data | contains three | entries, with the earliest |
| and its interpretation | columns: ‘full_text’ (the | post dated Mar. 5, |
| using DSM-5. Your task | content posted | 2020, and the latest |
| is to analyze the | by the user), ‘datetime’ | post dated May 14, |
| content | (the date and | 2020. The temporal |
| posted by the user, | time when the content | information extracted |
| specifically focusing on | was posted), | from the ‘Timeline’ |
| temporal information | and ‘Timeline’ | column and the |
| extracted | (temporal information | ‘full_text’ content is |
| from the ‘Timeline’ | extracted from the | summarized below in |
| column and | content).\n Summa- | chronological order: |
| corresponding | rize the temporal | * At least 20 years |
| chronological patterns | information from the | (Mar. 5, 2020): The |
| from | ‘Timeline’ column and | user mentions |
| the ‘datetime’ column. | arrange it in | struggling with |
| Ensure to summarize | chronological order | depression for at least |
| the timeline information | based on both the | 20 years. |
| in chronological order, | ‘datetime’ and the | * Years on years on |
| both from the content of | content's temporal | years on years (Apr. |
| the post and the | information. | 17, 2020): The user |
| timeline of | | mentions experiencing |
| when the user posted | | depression for multiple |
| it, and match it with | | years. |
| relevant DSM-5 criteria | | * Roughly year 20 (Apr. |
| covering | | 22, 2020): The user |
| every aspect in 500 | | confirms struggling with |
| words at the max. | | depression for |
| Output the summary in | | approximately 20 |
| a structured | | years. |
| format with: \n -Concise | | * Three years (May 14, |
| and Comprehensive | | 2020): The user |
| summary of timeline of | | mentions living for |
| posts | | others and getting |
| combining the in-text | | worried about |
| temporal features from | | themselves for the past |
| the ‘Timeline’ column | | three years. |
| and the | | * 15 years (May 14, |
| reporting date and time | | 2020): The user |
| from the ‘datetime’ | | mentions trying to |
| column and the | | escape reality and |
| corresponding | | feeling “fucked” for 15 |
| context from the | | years.” |
| ‘full_text’ column, for | | “Temporal patterns”: |
| instance, it carefully | | “The temporal patterns |
| notes every | | observed from the |
| temporal information | | ‘Timeline’ column and |
| giving us which incident | | the ‘full_text’ content |
| happen when and | | are: |
| when | | * Duration: The user |
| was it reported and | | has been struggling |
| what is the timeline | | with depression for at |
| information mentioned | | least 20 years, with |
| by the | | some periods lasting |
| user in the text in the | | for multiple years. |
| chronological order and | | * Frequency: The user |
| cross checking the | | mentions experiencing |
| timeline | | depression for multiple |
| information with the | | years, with no specific |
| content to know the | | mention of frequency. |
| reason of timeline | | * Recurrence: The |
| mentioning.\n | | user's symptoms seem |
| - Temporal patterns | | to have recurred over |
| observed (duration, | | the years, with no clear |
| frequency, recurrence, | | pattern of remission. |
| specific | | * Specific mentions of |
| mentions of time within | | time: The user |
| the text taking from | | mentions specific time |
| Timeline column and | | periods, such as 20 |
| the date and time it was | | years, 15 years, and 3 |
| posted). Limit your | | years, indicating a |
| output length to 500 | | sense of duration and |
| words. | | chronicity.”} |

At step 212 of the method of the present disclosure, the one or more hardware processors 104 generate, by using the one or more LLMs, a third summary (e.g., diagnosis summary and may be interchangeably used herein) based on the first summary and the second summary. The diagnosis summary for a particular user is extracted using the designed prompts that take temporal and non-temporal summary as an input for the same user. The prompts are provided below in Table 12 along with the example's diagnosis for Author1.

TABLE 12

| | | System | User | |
|---|---|---|---|---|
| | | Prompt for | Prompt for | |
| | | Diagnosis | Diagnosis | Diagnosis |
| Input | | Summary | Summary | Summary |
| Non- | Temporal | You are an | Summary: | Based on the |
| Temporal | Summary for | advanced | Dataframe.\n | provided data, |
| Summary for | Author1 | language | Review the | smarmyslayer, |
| Author1 | | model | provided | a Reddit user, |
| | | trained to | data of the | is experiencing |
| | | analyze and | user's overall | a moderate to |
| | | summarize | mental state | severe mental |
| | | text data | covering | health |
| | | related to | several | condition. The |
| | | mental state | features. | primary |
| | | of a | Summarize | causes of their |
| | | particular | this further | distress are |
| | | user. Given | concisely | loneliness, |
| | | a data | and provide | social |
| | | covering | the summary | isolation, and |
| | | various | in 400 words | academic |
| | | features of a | covering | struggles. |
| | | user based | every aspect | Their |
| | | on the | with clear | language style |
| | | content | information | is confessional |
| | | posted by | such that | and melancholic, |
| | | him/her on | when the | indicating a |
| | | reddit, | summary is | high level of |
| | | your task is | given to | emotional |
| | | to further | a mental | vulnerability. |
| | | summarize it | health | The recurring |
| | | concisely | practitioner | themes in |
| | | and provide | who works in | their posts |
| | | the summary | accordance | revolve |
| | | limited to | with DSM-5, | around |
| | | 400 words | it becomes | feelings of |
| | | covering | helpful for | loneliness, |
| | | every aspect | him/her to | social |
| | | with clear | recommend the | isolation, and |
| | | information | therapy and | anxiety, which |
| | | such that | behavior | are |
| | | when the | changes to the | characteristic |
| | | summary is | user based | of Major |
| | | given to a | on it. Your | Depressive |
| | | mental health | task is | Disorder |
| | | practitioner | specified to | (MDD) as |
| | | who works in | generate | outlined in the |
| | | accordance | such a | DSM-5. The |
| | | with DSM-5, | summary | user's |
| | | it becomes | which covers | symptoms |
| | | helpful for | all the | meet the |
| | | him/her to | necessary | criteria for |
| | | recommend | information | MDD, with |
| | | the therapy | regarding | persistent |
| | | and behavior | the mental | feelings of |
| | | changes to | health state | sadness, |
| | | the user | of the user. | hopelessness, |
| | | based on it. | | and a lack of |
| | | Your task is | | interest in |
| | | specified | | activities they |
| | | to generate | | once enjoyed. |
| | | such a | | Their mental |
| | | summary | | health status is |
| | | which covers | | characterized |
| | | all the | | by a pervasive |
| | | necessary | | sense of sadness, |
| | | information | | anxiety, and a |
| | | regarding the | | sense of |
| | | mental | | disconnection |
| | | health state | | from others. |
| | | of the user. | | The user's |
| | | | | posts suggest |
| | | | | a chronic and |
| | | | | debilitating |
| | | | | condition that |
| | | | | has been |
| | | | | ongoing for an |
| | | | | extended period. |
| | | | | The temporal |
| | | | | patterns |
| | | | | observed in |
| | | | | the user's |
| | | | | posts indicate |
| | | | | a consistent |
| | | | | pattern of |
| | | | | distress over a |
| | | | | four-month |
| | | | | period, with |
| | | | | no clear |
| | | | | indication of |
| | | | | improvement |
| | | | | or worsening. |
| | | | | The duration |
| | | | | of their |
| | | | | symptoms is |
| | | | | concerning, as |
| | | | | it suggests a |
| | | | | prolonged |
| | | | | period of |
| | | | | suffering that |
| | | | | may be |
| | | | | impacting their |
| | | | | daily life and |
| | | | | relationships. |
| | | | | A mental health |
| | | | | practitioner |
| | | | | working in |
| | | | | accordance |
| | | | | with the DSM- |
| | | | | 5 would likely |
| | | | | diagnose |
| | | | | smarmyslayer |
| | | | | with Major |
| | | | | Depressive |
| | | | | Disorder and |
| | | | | recommend |
| | | | | evidence- |
| | | | | based |
| | | | | treatments |
| | | | | such as |
| | | | | cognitive- |
| | | | | behavioral |
| | | | | therapy and |
| | | | | medication to |
| | | | | alleviate their |
| | | | | symptoms. |

At step 214 of the method of the present disclosure, the one or more hardware processors 104 generate, by using the one or more LLMs, one or more actionable health disorder specific recommendations based on an analysis of the third summary. The one or more actionable health disorder specific recommendations comprise at least one of a therapy, and one or more behavioral changes. For users dealing with daily stressors or issues stemming from lifestyle factors, the system 100 recommends practical behavioral changes. These include, for example, but are not limited to, adopting mindfulness techniques to enhance emotional regulation and stress management, engaging in regular physical exercise to improve overall mental and physical health, and implementing strategies to improve sleep patterns to ensure restorative rest. By providing these comprehensive recommendations, we aim to offer the user a balanced approach that combines professional support with actionable lifestyle modifications, thereby fostering long-term mental health and overall well-being. Table 13 depicts exemplary recommendations generated by the system 100 by using the third summary (e.g., the diagnosis summary):

TABLE 13

| | System Prompt for | User Prompt for | |
|---|---|---|---|
| Input | Recommendations | Recommendations | Recommendations |
| Based on the | You are a Mental | Summary: | Therapy |
| provided data, | Health Practitioner | Dataframe.\n | Recommendations: |
| smarmyslayer, | working in | Review the | 1. Cognitive |
| a Reddit | accordance with | provided summary | Behavioral |
| user, is | DSM-5. | of the user's | Therapy (CBT) |
| experiencing | Given the | behavior and | 2. Interpersonal |
| a moderate | summary of a | mental health. | Therapy (IPT) |
| to severe | user's behavior | Consider all the | 3. Psychodynamic |
| mental | and mental health | important | Therapy |
| health | trends, provide | information, and | Behavior |
| condition. | therapy | generate at | Modification |
| The primary | recommendations | the max three | Suggestions: |
| causes of | and suggest | most suitable | 1. Engage in |
| their distress | behavior changes | therapy | regular physical |
| are loneliness, | separately that | recommendations | activity to improve |
| social isolation, | could benefit the | and five | mood and reduce |
| and academic | user. Consider all | actionable | symptoms of |
| struggles. | the relevant and | behavior | depression. |
| Their | important | modification | 2. Practice social |
| language | information | suggestions | skills and connect |
| style is | mentioned in the | separately that | with others |
| confessional | summary and | could help the | through online |
| and | recommend at the | user improve | communities or in- |
| melancholic, | max three most | their well-being. | person support |
| indicating a | suitable | Do not provide | groups. |
| high level of | therapies and five | any extra | 3. Set realistic |
| emotional | actionable | explanations. | goals and |
| vulnerability. | behavior changes | | prioritize self-care |
| The recurring | accordingly. | | activities to |
| themes in | | | increase sense of |
| their posts | | | accomplishment |
| revolve | | | and purpose. |
| around | | | 4. Limit social |
| feelings of | | | media use and |
| loneliness, | | | avoid comparing |
| social | | | oneself to others |
| isolation, and | | | to reduce feelings |
| anxiety, | | | of inadequacy and |
| which are | | | isolation. |
| characteristic | | | 5. Keep a daily |
| of Major | | | journal to process |
| Depressive | | | emotions and |
| Disorder | | | identify patterns |
| (MDD) as | | | and triggers of |
| outlined in | | | distress. |
| the DSM-5. | | | |
| The user's | | | |
| symptoms | | | |
| meet the | | | |
| criteria for | | | |
| MDD, with | | | |
| persistent | | | |
| feelings of | | | |
| sadness, | | | |
| hopelessness, | | | |
| and a lack | | | |
| of interest in | | | |
| activities | | | |
| they once | | | |
| enjoyed. | | | |
| Their mental | | | |
| health status is | | | |
| characterized | | | |
| by a pervasive | | | |
| sense of | | | |
| sadness, | | | |
| anxiety, and | | | |
| a sense of | | | |
| disconnection | | | |
| from others. The | | | |
| user's posts | | | |
| suggest a | | | |
| chronic and | | | |
| debilitating | | | |
| condition that | | | |
| has been | | | |
| ongoing for | | | |
| an extended | | | |
| period. The | | | |
| temporal | | | |
| patterns | | | |
| observed in | | | |
| the user's | | | |
| posts | | | |
| indicate a | | | |
| consistent | | | |
| pattern of | | | |
| distress over | | | |
| a four-month | | | |
| period, with | | | |
| no clear | | | |
| indication of | | | |
| improvement or | | | |
| worsening. | | | |
| The duration | | | |
| of their | | | |
| symptoms is | | | |
| concerning, as | | | |
| it suggests a | | | |
| prolonged | | | |
| period of | | | |
| suffering that | | | |
| may be | | | |
| impacting | | | |
| their daily | | | |
| life and | | | |
| relationships. | | | |
| A mental | | | |
| health | | | |
| practitioner | | | |
| working in | | | |
| accordance | | | |
| with the | | | |
| DSM-5 | | | |
| would likely | | | |
| diagnose | | | |
| smarmyslayer | | | |
| with Major | | | |
| Depressive | | | |
| Disorder and | | | |
| recommend | | | |
| evidence- | | | |
| based | | | |
| treatments | | | |
| such as | | | |
| cognitive- | | | |
| behavioral | | | |
| therapy and | | | |
| medication to | | | |
| alleviate their | | | |
| symptoms. | | | |

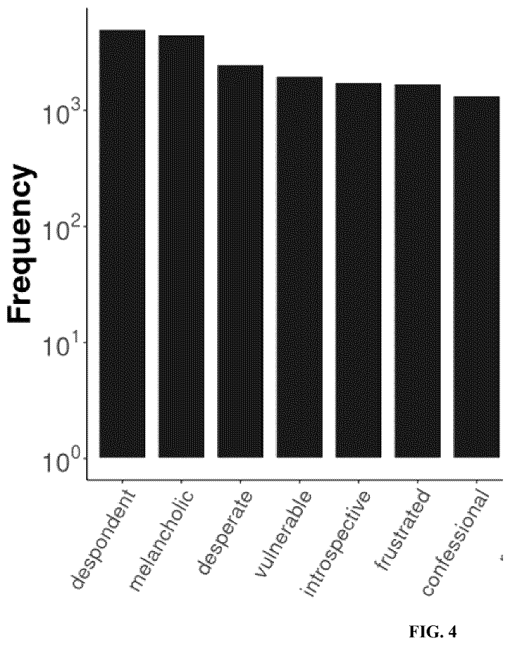

FIG. 4, with reference to FIGS. 1 through 3, depicts a graphical representation illustrating used language styles and tones by online users, in accordance with an embodiment of the present disclosure. The frequency of these language styles and tones provides valuable insights into the emotional landscape of users within the dataset. The prominence of negative tones like despondent and melancholic aligns with the high occurrence of depressive disorders and related mental health concerns, demonstrating a strong correlation between language use and the emotional or psychological state of users.

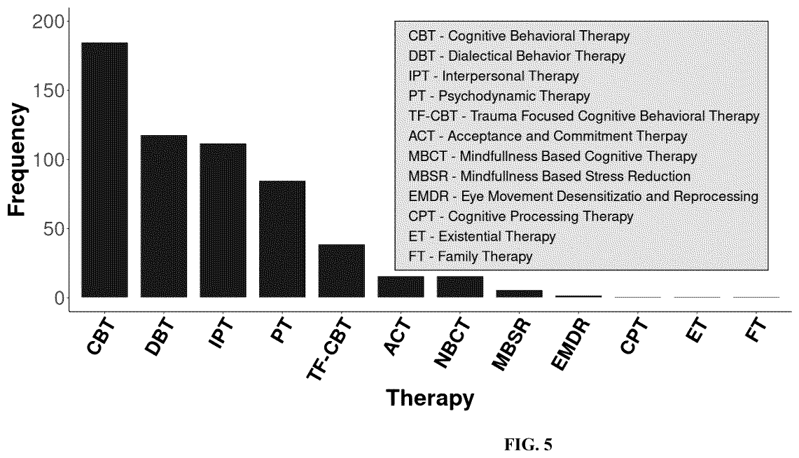

FIG. 5, with reference to FIGS. 1 through 4, depicts a graphical representation illustrating distribution of various therapies recommended to the users, in accordance with an embodiment of the present disclosure. It is observed that Cognitive Behavioral Therapy (CBT) emerged as the most common recommendation, highlighting its widespread applicability for various mental health issues. Following closely, Dialectical Behavior Therapy (DBT) was recommended, indicating its effectiveness in addressing emotional regulation and interpersonal effectiveness. Additionally, Interpersonal Therapy (IPT) was suggested, reflecting its relevance in improving social functioning and focusing on enhancing interpersonal relationships. Beyond these three, several other therapies were also recommended, demonstrating a diverse array of interventions tailored to meet the specific needs of users based on their diagnoses. This breadth of recommendations underscores the importance of personalized treatment approaches in mental health care.

In the present disclosure, embodiments provide a system and a method for the evaluation of multiple LLMs involving binary, multi-class, multi-label classification, and recommendation tasks from social media discussions related to mental health. Extracting domain-specific features from social media data is challenging due to its unstructured nature, making it difficult to identify relevant information accurately. Temporal information in text is often sparse, requiring a specialized approach for accurate capture. LLMs frequently overlook this, resulting in incomplete user profile summaries. By combining text creation time with temporal details extracted from the content, the system can build a chronological mental health history, which is essential for DSM-5 diagnosis. Collecting a user's text across various timelines and providing it to LLMs poses a challenge due to input token limitations, making it hard to include all relevant details. To address this, the system extracted key features from the text and used these features to create a comprehensive mental health summary for each user, ensuring important information is retained. The method of the present disclosure is not only robust in identifying a wide range of mental health disorders but also versatile enough to provide tailored recommendations for therapy and behavior changes. Although the system and method of the present disclosure present results for a sample dataset, the framework can be scaled up for much larger datasets, making it a valuable tool for large-scale mental health assessment and recommendations.

Moreover, careful prompt engineering of LLMs has proven effective in generating comprehensive insights, including disorder identification, severity assessment, recommendation of therapy and behavior changes. The experimental results were conducted as depicted in the graphs of FIGS. 4 and 5 through various LLMs being implemented by the system as known in the art. It is to be understood by a person having ordinary skill in the art or person skilled in the art various prompts may be provided to the LLMs depending upon their configurations and such prompts shall not be construed as limiting the scope of the present disclosure.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor implemented method, comprising:
receiving, via one or more hardware processors, an input data pertaining to a plurality of users;
filtering, by using one or more Large Language Models (LLMs) via the one or more hardware processors, the input data to obtain a filtered data specific to at least one health disorder pertaining to at least a subset of the plurality of users;
extracting, by using the one or more LLMs via the one or more hardware processors, a first set of features and a second set of features from the filtered data based on one or more context associated prompts;
creating, by using the one or more LLMs via the one or more hardware processors, one or more user profiles for the subset of the plurality of users based on the extracted first set of features and the extracted second set of features;
generating a first summary and a second summary based on the extracted first set of features and the extracted second set of features respectively for each user profile amongst the one or more user profiles;
generating, by using the one or more LLMs via the one or more hardware processors, a third summary based on the first summary and the second summary; and
generating, by using the one or more LLMs via the one or more hardware processors, one or more actionable health disorder specific recommendations based on an analysis of the third summary.

2. The processor implemented method of claim 1, wherein the first set of features comprises at least one of (i) a duration, (ii) a frequency, (iii) a recurrence, and (iv) one or more explicit time and date mentions.

3. The processor implemented method of claim 1, wherein the second set of features comprises at least one of (i) a severity of the at least one health disorder, (ii) one or more factors impacting the at least one health disorder, and (iii) a language and tonality in the filtered data pertaining to the at least one health disorder.

4. The processor implemented method of claim 1, wherein the one or more actionable health disorder specific recommendations comprise at least one of a therapy, and one or more behavioral changes.

5. A system, comprising:
a memory storing instructions;
one or more communication interfaces; and
one or more hardware processors coupled to the memory via the one or more communication interfaces, wherein the one or more hardware processors are configured by the instructions to:
receive an input data pertaining to a plurality of users;
filter, by using one or more Large Language Models (LLMs), the input data to obtain a filtered data specific to at least one health disorder pertaining to at least a subset of the plurality of users;
extract, by using the one or more LLMs, a first set of features and a second set of features from the filtered data based on one or more context associated prompts;
create, by using the one or more LLMs, one or more user profiles for the subset of the plurality of users based on the extracted first set of features and the extracted second set of features;
generate, by using the one or more LLMs, a first summary and a second summary based on the extracted first set of features and the extracted second set of features respectively for each user profile amongst the one or more user profiles;
generate, by using the one or more LLMs, a third summary based on the first summary and the second summary; and
generate, by using the one or more LLMs, one or more actionable health disorder specific recommendations based on an analysis of the third summary.

6. The system of claim 5, wherein the first set of features comprises at least one of (i) a duration, (ii) a frequency, (iii) a recurrence, and (iv) one or more explicit time and date mentions.

7. The system of claim 5, wherein the second set of features comprises at least one of (i) a severity of the at least one health disorder, (ii) one or more factors impacting the at least one health disorder, and (iii) a language and tonality in the filtered data pertaining to the at least one health disorder.

8. The system of claim 5, wherein the one or more actionable health disorder specific recommendations comprise at least one of a therapy, and one or more behavioral changes.

9. One or more non-transitory machine-readable information storage mediums comprising one or more instructions which when executed by one or more hardware processors cause:
receiving input data pertaining to a plurality of users;
filtering by using one or more Large Language Models (LLMs) the input data to obtain a filtered data specific to at least one health disorder pertaining to at least a subset of the plurality of users;
extracting by using the one or more LLMs, a first set of features and a second set of features from the filtered data based on one or more context associated prompts;
creating by using the one or more LLMs, one or more user profiles for the subset of the plurality of users based on the extracted first set of features and the extracted second set of features;
generating a first summary and a second summary based on the extracted first set of features and the extracted second set of features respectively for each user profile amongst the one or more user profiles;
generating by using the one or more LLMs, a third summary based on the first summary and the second summary; and
generating by using the one or more LLMs, one or more actionable health disorder specific recommendations based on an analysis of the third summary.

10. The one or more non-transitory machine-readable information storage mediums of claim 9, wherein the first set of features comprises at least one of (i) a duration, (ii) a frequency, (iii) a recurrence, and (iv) one or more explicit time and date mentions.

11. The one or more non-transitory machine-readable information storage mediums of claim 9, wherein the second set of features comprises at least one of (i) a severity of the at least one health disorder, (ii) one or more factors impacting the at least one health disorder, and (iii) a language and tonality in the filtered data pertaining to the at least one health disorder.

12. The one or more non-transitory machine-readable information storage mediums of claim 9, wherein the one or more actionable health disorder specific recommendations comprise at least one of a therapy, and one or more behavioral changes.
